SEQUENCE LISTING (4 sequences)

SEQ ID NO: 1 - DNA, 1972 bp, Artificial Sequence
  cDNA of sVEGFRv-1
caccatggtcagctactgggacaccggggtcctgctgtgcgcgctgctcacctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcaga

SEQ ID NO: 2 - protein, 656 aa, Artificial Sequence
  sVEGFRv-1
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIR

SEQ ID NO: 3 - DNA, 33 bp, Artificial Sequence
  sVEGFRv-1 F1 primer
aaggtaccgccaccatggtcagctactgggaca

SEQ ID NO: 4 - DNA, 35 bp, Artificial Sequence
  sVEGFRv-1 R3 primer
cgctcgagctatctgattgtaatttctttcttctg

(19) United States
(12) Patent Application Publication
(10) Pub. No.: US 2023/0024569 A1
(43) Pub. Date: Jan. 26, 2023

(54) COMPOSITION FOR TREATING DIABETIC RETINOPATHY, COMPRISING RAAV CONTAINING SOLUBLE VEGFR-1 VARIANT CDNA

(71) Applicant: CdmoGen Co., Ltd., Chungcheongbuk-do (KR)

(72) Inventors: Steven Hyun Seung LEE, Chungcheongbuk-do (KR); Kee Rang PARK, Chungcheongbuk-do (KR); Young-lll LEE, Chungcheongbuk-do (KR)

(21) Appl. No.: 17/781,960

(22) PCT Filed: Dec. 3, 2020
(86) PCT No.: PCT/KR2020/017570

§ 371 Date: Jun. 2, 2022

(30) Foreign Application Priority Data
Dec. 4, 2019 (KR) 10-2019-0159982

(51) Int. Cl. A61K 48/00; A61P 27/02

(52) U.S. Cl. CPC A61K 48/005; A61P 27/02; A61K 48/0083

(57) ABSTRACT

The present invention provides a pharmaceutical composition for treating diabetic retinopathy. According to the present invention, the pharmaceutical composition can treat diabetic retinopathy by means of a single administration and thus, compared to conventional treatment methods, which require intraocular injection on a monthly basis, can reduce pain and ocular damage and infections in patients and reduce treatment costs.

### TECHNICAL FIELD

The present invention relates to a pharmaceutical composition for treating diabetic retinopathy, and more particularly to a pharmaceutical composition for treating diabetic retinopathy including a recombinant vector containing a cDNA of a soluble VEGF receptor variant.

### BACKGROUND ART

Diabetic retinopathy is one of three major retinal diseases, along with glaucoma and macular degeneration. Diabetic retinopathy, which is a retinal disease caused by high blood glucose, is a disease in which retinal vascular degeneration is induced, and retinal edema, exudate deposition and hemorrhage due to vascular leakage, and retinal neurodegeneration occur. Diabetic retinopathy is divided into proliferative diabetic retinopathy and nonproliferative diabetic retinopathy, and there is also diabetic macular edema.

For current treatment of diabetic retinopathy, methods of treating retinal edema and proliferative retinopathy are mainly used. Retinal edema is treated through injection of anti-VEGF, and photocoagulation using a laser is performed in cases of vascular proliferation or bleeding.

Meanwhile, gene therapy is a method of treating a disease through gene delivery and expression, and corrects a genetic defect by targeting a specific gene with a disorder, unlike drug therapy. The ultimate goal of gene therapy is to obtain beneficial therapeutic effects by genetically modifying living cells. This therapy is receiving attention as the best treatment for disease because of advantages of accurate delivery of genetic factors to disease sites, complete degradation in vivo, absence of toxicity and immune antigenicity, and stable long-term expression of genetic factors.

Gene therapy includes methods of introducing a gene having a therapeutic effect on a specific disease, enhancing the resistance functionality of normal cells to exhibit resistance to anticancer drugs, etc., or replacing modified or lost genes in patients of various hereditary diseases.

Gene therapy is largely divided into two types, namely in-vivo gene therapy and ex-vivo gene therapy. In-vivo gene therapy includes directly injecting a therapeutic gene into the body, and ex-vivo gene therapy includes primarily culturing target cells in vitro, introducing a gene into the cells, and then injecting the genetically modified cells into the body. Currently, in the field of gene therapy research, ex-vivo gene therapy is more widely used than in-vivo gene therapy.

Gene delivery technology may be broadly classified into a method using a virus as a carrier (viral vector-based transfer method), a nonviral delivery method using a synthetic phospholipid or synthetic cationic polymer, and a physical method such as electroporation for introducing a gene by applying temporary electrical stimulation to the cell membrane.

In gene delivery technology, viruses used as viral carriers or viral vectors include RNA viral vectors (retroviral vectors, lentiviral vectors, etc.) and DNA viral vectors (adenoviral vectors, adeno-associated viral vectors, etc.). In addition thereto, the viruses include herpes simplex viral vectors, vaccinia virus vectors, alpha viral vectors, and the like.

Meanwhile, an adenovirus, which is a cloning vector, is advantageous in that it may be replicated to have an intermediate size in the cell nucleus, is clinically non-toxic, is stable even when an external gene is inserted, does not cause gene rearrangement or loss, is able to transform eukaryotes, and is expressed stably at high levels even when integrated into host cell chromosomes. Host cells suitable for adenovirus are cells causative of human hematopoiesis, lymph, and myeloma. However, since adenovirus is linear DNA, it is difficult to culture, it is not easy to recover an infected virus, and the rate of infection with the virus is low. Moreover, expression of the delivered gene is greatest after 1 to 2 weeks, and in some cells, expression is maintained for only 3 to 4 weeks. The high immunogenicity thereof is also problematic.

An adeno-associated virus (AAV) has recently been favored because it is able to overcome the problems described above and has many advantages as a gene therapeutic agent. AAV is a single-stranded provirus and requires a helper virus for replication, and the AAV genome is 4,680 bp in length and may be inserted at a specific site at chromosome 19 of an infected cell. A transgene is inserted into plasmid DNA linked by two inverted terminal repeat (ITR) sequences of 145 bp each in length and a signal sequence. In order to produce an AAV-based gene therapeutic agent, another plasmid DNA expressing the AAV rep part and the cap part is additionally transfected therewith, and at the same time, adenovirus, which is absolutely necessary for AAV amplification, is directly added as a helper virus or is added in the form of a separate plasmid containing an adenovirus gene (E1, E4, or VA RNA gene) responsible for an important function in amplification.

AAV has advantages in that it delivers a gene to a wide range of host cells, causes few immune side effects upon repeated administration, and maintains gene expression for several years. Moreover, the AAV genome is safe even when integrated into the chromosomes of host cells, and does not cause modification or rearrangement of gene expression in host cells.

A vascular endothelial growth factor (VEGF) acts by binding to an fms-like tyrosine kinase receptor Flt-1 and paired KDR/Flk-1, and interaction with the latter induces an angiogenic process. Soluble Flt-1 (sFLT-1) is a soluble variant that does not have a membrane-spanning domain present in Flt-1, and naturally occurs through a selective splicing process. sFlt-1 binds directly to VEGF with high affinity to thus prevent VEGF from interacting with KDR/Flk-1, thereby exhibiting anti-VEGF activity (anti-angiogenic activity) (McLeod et al., Investigative Ophthalmology & Visual Science, 43:474, 2002). sFlt-1 also binds to KDR/Flk-1 to form an inactive heterodimer. Since this anti-VEGF activity functions as anti-angiogenic activity, an antibody and a fusion protein taking advantage of anti-VEGF activity are currently used as therapeutic agents for retinal degeneration (Simo et al., Diabetes Care, 37:893, 2014), and examples thereof include Lucentis (ranibizumab) developed by Genentech and sold by Novartis, Eylea (aflibercept) developed by Regeneron Pharmaceuticals, and Avastin (bevacizumab). However, these protein therapeutic agents have to be intravitreally injected once every 1-2 months, causing serious side effects. Hence, it is urgently necessary to develop a therapeutic agent for macular degeneration that is fundamentally capable of exhibiting long-term therapeutic efficacy through a single injection.

Accordingly, the present inventors have made great efforts to develop a therapeutic agent capable of treating diabetic retinopathy through only a single administration, developed a soluble VEGF receptor variant including a new region rather than the naturally occurring soluble VEGF receptor studied to date, and ascertained that, when a gene therapeutic agent packaged in an adeno-associated viral vector (AAV) is administered to a diabetic retinopathy mouse model, symptoms of diabetic retinopathy, such as retinal macular edema, vascular leakage, and vascular degeneration, are inhibited, thus culminating in the present invention.

### DISCLOSURE

It is an object of the present invention to provide a composition for treating diabetic retinopathy capable of treating diabetic retinopathy through only a single administration.

It is another object of the present invention to provide a method of treating or preventing diabetic retinopathy including administering a recombinant vector containing a cDNA of a soluble VEGF receptor variant (sVEGFRv-1 cDNA).

It is still another object of the present invention to provide the use of the recombinant vector containing the cDNA of soluble VEGF receptor variant (sVEGFRv-1 cDNA) for the treatment or prevention of diabetic retinopathy.

It is yet another object of the present invention to provide the use of the recombinant vector containing the cDNA of soluble VEGF receptor variant (sVEGFRv-1 cDNA) for the manufacture of a medicament for the treatment or prevention of diabetic retinopathy.

In order to accomplish the above objects, the present invention provides a pharmaceutical composition for treating or preventing diabetic retinopathy including a recombinant vector containing a cDNA of a soluble VEGF receptor variant (sVEGFRv-1 cDNA).

In addition, the present invention provides a method of treating or preventing diabetic retinopathy including administering the recombinant vector containing the cDNA of soluble VEGF receptor variant (sVEGFRv-1 cDNA).

In addition, the present invention provides the use of the recombinant vector containing the cDNA of soluble VEGF receptor variant (sVEGFRv-1 cDNA) for the treatment or prevention of diabetic retinopathy.

In addition, the present invention provides the use of the recombinant vector containing the cDNA of soluble VEGF receptor variant (sVEGFRv-1 cDNA) for the manufacture of a medicament for the treatment or prevention of diabetic retinopathy.

### DESCRIPTION OF DRAWINGS

FIG. 1 shows the configuration of a soluble VEGF receptor variant inserted into rAAV2-sVEGFRv-1 used in the present invention in comparison with sFlt-1, which is a naturally occurring soluble VEGF receptor;

FIG. 2 shows images confirming expression of the soluble VEGF receptor variant in the eye after intravitreal injection of rAAV2-sVEGFRv-1 used in the present invention (A) and a graph of the amount of the soluble VEGF receptor variant secreted into the vitreous body (B);

FIG. 3 shows images (A) for observation of retinal vascular leakage using dextran-FITC in a diabetic retinopathy mouse model and an analysis graph thereof (B), in which retinal images of a control group, a positive control group, and a group administered with rAAV2-sVEGFRv-1 are shown, indicating that vascular leakage was inhibited by administration of rAAV2-sVEGFRv-1;

FIG. 4 shows TUNEL-stained images (A) for observation of retinal neurodegeneration in a diabetic retinopathy mouse model and an analysis graph thereof (B), in which retinal images of a control group, a positive control group, and a group administered with rAAV2-sVEGFRv-1 are shown, indicating that retinal neurodegeneration was inhibited by administration of rAAV2-sVEGFRv-1 compared to the control groups;

FIG. 5 shows NeuN-stained images (A) for observation of retinal ganglion cells in a diabetic retinopathy mouse model and an analysis graph thereof (B), in which retinal images of a negative control group, a positive control group, and a group administered with rAAV2-sVEGFRv-1 are shown, indicating that degeneration of retinal ganglion cells was inhibited by administration of rAAV2-sVEGFRv-1 compared to the control groups;

FIG. 6 shows GFAP-stained images (A) for observation of glial cell activity in a diabetic retinopathy mouse model and an analysis graph thereof (B), in which retinal images of a control group, a positive control group, and a group administered with rAAV2-sVEGFRv-1 are shown, indicating that the extent of staining with GFAP was decreased by administration of rAAV-sVEGFRv-1 compared to the control groups and also that the activity of glial cells was inhibited by administration of the therapeutic vector;

FIG. 7 shows images (A) of isolated retinal blood vessels for observation of retinal vascular degeneration in a diabetic retinopathy mouse model and an analysis graph thereof (B), in which retinal images of a control group, a positive control group, and a group administered with rAAV2-sVEGFRv-1 are shown, indicating that loss of pericytes and generation of acellular capillaries in retinal blood vessels were inhibited by administration of rAAV2-sVEGFRv-1 compared to the control groups; and

FIG. 8 shows images (A) confirming the degeneration and protective effect of retinal tissue after staining of the retinal tissue in a diabetic retinopathy mouse model and an analysis graph thereof (B), in which retinal images of a control group, a positive control group, and a group administered with rAAV2-sVEGFRv-1 are shown, indicating that the retinal tissue was thickened by administration of rAAV2-sVEGFRv-1 compared to the control groups and also confirming the effect of protection of retinal nerves due to administration of rAAV2-sVEGFRv-1.

### MODE FOR INVENTION

Unless otherwise defined, all technical and scientific terms used herein have the same meanings as those typically understood by those skilled in the art to which the present invention belongs. In general, the nomenclature used herein is well known in the art and is typical.

In diabetic retinopathy, vascular leakage, neurodegeneration, and angiogenesis are induced by the action of VEGF caused by high blood glucose. The present invention intends to develop a gene therapeutic agent for inhibiting the action of expressed VEGF and to inhibit the expression of VEGF by expressing the soluble VEGF receptor 1.

In the present invention, in order to inhibit diabetic retinopathy, a therapeutic agent for delivering a therapeutic gene to the retina by loading the therapeutic gene on a recombinant adeno-associated virus (AAV) was developed, and the effects thereof were confirmed using an animal model.

In the present invention, in order to inhibit diabetic retinopathy caused by angiogenesis, rAAV2-soluble VEGFRv-1 (rAAV2-sVEGFRv-1), which is a candidate for a soluble VEGFRv-1 gene therapeutic agent, was developed to block the activity of VEGF in the retina by loading a soluble VEGFR-1 variant therapeutic gene on a recombinant adeno-associated virus (AAV), and the therapeutic efficacy thereof was confirmed using a diabetic-retinopathy-induced animal model. In addition, the rAAV2-soluble VEGFRv-1 gene therapeutic agent was confirmed to exhibit superior efficacy compared to the therapeutic efficacy of existing antibody-based drugs such as Avastin and bevacizumab.

Accordingly, an aspect of the present invention relates to a pharmaceutical composition for treating or preventing diabetic retinopathy including a recombinant vector containing a cDNA of a soluble VEGF receptor variant.

The cDNA of soluble VEGF receptor variant used in the present invention is a fragment of the spanning nucleotide position 282-2253 in a human vascular endothelial growth factor receptor (VEGFR) 1 gene (XM_017020485.1, NCBI reference sequence, NIH), and has a total size of 1,972 bp (SEQ ID NO: 1), amino acids expressed from the 5th base of SEQ ID NO: 1, and have the amino acid sequence of SEQ ID NO: 2.

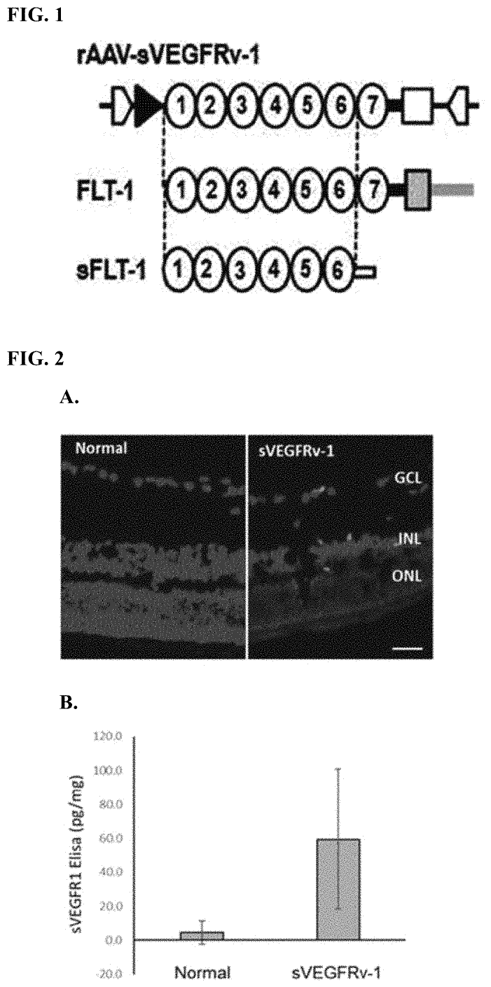

As shown in FIG. 1A, the soluble VEGF receptor variant is a variant having 6 immunoglobulin-like domains having binding sites with VEGF and PIGF and 31 N-termini identical to FLT-1, and sFLT-1, which is a naturally occurring variant of FLT-1, has 6 immunoglobulin-like domains and a tail composed of 37 amino acids different from FLT-1, and thus the variants have configurations different from each other (Kendall, R. L. and Thomas, K. A. Proc. Natl. Acad. Sci. USA 90, 10705-10709, 1993).

In the present invention, the recombinant vector may be an adeno-associated virus, preferably rAAV2 or another rAAV serotype.

In an embodiment of the present invention, in order to construct rAAV2-sVEGFRv-1, which is a recombinant vector containing a soluble VEGF receptor variant (sVEGFRv-1) cDNA, pAAV-sVEGFRv-1 was produced by inserting sVEGFRv-1 cDNA into a pAAV-F.IX cis plasmid containing a CMV promoter, an SV40 polyadenylation signal, and two ITRs (U.S. Pat. No. 6,093,392), and all of pAAV-sVEGFRv-1, pAAV-R2C2, and pHelper were transfected into HEK293 (ATCC CRL-1573) cells, followed by culture for 72 hours, after which the HEK293 cells were harvested and sonicated, and recombinant AAV (rAAV) particles were subjected to CsCl density gradient centrifugation two times. In the first, a fraction of 1.37 to 1.42 g/mL was collected and separated, and in the second, a fraction of 1.35 to 1.43 g/mL was collected and purely separated to obtain a rAAV2-sVEGFRv-1 vector.

Therefore, the composition for treating diabetic retinopathy according to the present invention is an AAV-based gene therapeutic agent capable of overcoming problems with existing anti-VEGF therapeutic agents such as the pain associated with repeated monthly intravitreal injection, side effects such as ocular damage and infection due to injections, high treatment costs, and the like.

In the present invention, the effects of rAAV2-sVEGFRv-1 on inhibition of vascular leakage, inhibition of retinal neurodegeneration, inhibition of glial cell activity, and inhibition of retinal vascular degeneration were confirmed, indicating that rAAV2-sVEGFRv-1 of the present invention exhibits a therapeutic effect similar to that of bevacizumab, which is a known therapeutic agent for diabetic retinopathy. Moreover, the rAAV2-sVEGFRv-1 of the present invention is capable of exhibiting continuous therapeutic effects for 2-3 years through a single injection without the need for direct intravitreal injection every month, as with bevacizumab (Avastin), ranibizumab (Lucentis), etc.

The composition of the present invention may be administered once every 2-3 years.

In the present invention, the composition for treating diabetic retinopathy may be provided in the form of an injectable formulation, and the injectable formulation may further contain a stabilizer selected from the group consisting of Ethyl oleate, Polyvinylpyrrolidone (PVP10), Ganglioside Sodium L-lactate, Zinc chloride and Sucrose. In a preferred embodiment, 10 mg/ml of ethyl oleate, 1 mg/ml of PVP10, 0.1 mg/ml of GM1, 1 mg/ml of sodium L-lactate, 0.1 mg/ml of zinc chloride, or 10 mg/ml of sucrose in a HEPES buffer (20 mM, pH 7.4) is used.

In the present invention, the rAAV2-sVEGFRv-1 vector may be administered at 1×10⁵ to 1×10¹⁰ vg/eye, preferably 1×10⁸ to 1×10¹⁰ vg/eye, and more preferably 1×10⁸ to 1×10⁹ vg/eye.

Another aspect of the present invention relates to a method of treating or preventing diabetic retinopathy including administering the recombinant vector containing the cDNA of soluble VEGF receptor variant (sVEGFRv-1 cDNA).

Still another aspect of the present invention relates to a use of the recombinant vector containing the cDNA of soluble VEGF receptor variant (sVEGFRv-1 cDNA) for the treatment or prevention of diabetic retinopathy.

Yet another aspect of the present invention relates to a use of the recombinant vector containing the cDNA of soluble VEGF receptor variant (sVEGFRv-1 cDNA) for the manufacture of a medicament for the treatment or prevention of diabetic retinopathy.

Carriers useful in the pharmaceutical composition of the present invention include pharmaceutically acceptable carriers, adjuvants, and vehicles, collectively referred to as “pharmaceutically acceptable carriers”. Examples of pharmaceutically acceptable carriers that may be used in the pharmaceutical composition of the present invention include, but are not limited to, ion exchangers, alumina, aluminum stearate, lecithin, serum proteins (e.g. human serum albumin), buffer materials (e.g. several phosphates, glycine, sorbic acid, potassium sorbate, partial glyceride mixtures of saturated vegetable fatty acids), water, salts or electrolytes (e.g. protamine sulfate, disodium hydrogen phosphate, potassium hydrogen phosphate, sodium chloride, and zinc salts), colloidal silica, magnesium trisilicate, polyvinylpyrrolidone, cellulose-based substrates, polyethylene glycol, sodium carboxymethyl cellulose, polyarylate, waxes, polyethylene-polyoxypropylene-block polymers, polyethylene glycol, and wool fat.

The composition for treatment of the present invention is preferably administered parenterally. As used herein, the term “parenteral” includes subcutaneous, intradermal, intravenous, intramuscular, intraarticular, intrasynovial, intrasternal, intradural, intralesional, and intracranial injection or infusion techniques.

The pharmaceutical composition may be a sterile injectable aqueous or oily suspension in the form of a sterile injectable formulation. This suspension may be formulated according to techniques known in the art using suitable dispersants or wetting agents (e.g. Tween 80) and suspension agents. The sterile injectable formulation may also be a sterile injectable solution or suspension (e.g. a solution in 1,3-butanediol) in a non-toxic parenterally acceptable diluent or solvent. Vehicles and solvents that may be acceptably used include mannitol, water, Ringer's solution, and isotonic sodium chloride solution. In addition, sterile nonvolatile oil may be typically employed as a solvent or suspension medium. For this purpose, any less-stimulating nonvolatile oil including synthetic mono- or di-glycerides may be used. Fatty acids, such as oleic acid and glyceride derivatives thereof, may be used in injectable formulations like pharmaceutically acceptable natural oils (e.g. olive oil or castor oil), particularly polyoxyethylated forms thereof.

The composition of the present invention may be used in combination with a typical anti-inflammatory agent or in combination with a matrix metalloprotease inhibitor, a lipoxygenase inhibitor, and an inhibitor of cytokine other than IL-1β. The composition of the present invention may also be administered in combination with an immunomodulatory agent (e.g. bropirimine, anti-human alpha interferon antibody, IL-2, GM-CSF, methionine enkephalin, interferon alpha, diethyldithiocarbamate, tumor necrosis factor, naltrexone, and rEPO), or prostaglandin, in order to prevent or eliminate IL-1-mediated disease symptoms such as inflammation. When the composition of the present invention is administered in combination with other therapeutic agents, they may be administered to the patient sequentially or simultaneously. Alternatively, the pharmaceutical composition according to the present invention may be prepared by mixing the composition of general formula (1) with the other therapeutic or prophylactic agent described above.

The amount of the antibody that may be combined with the carrier material to produce a single dosage form may vary depending on the treated host and the particular mode of administration.

However, it will be understood that the specific effective amount for a particular patient may vary depending on various factors including the activity of the particular compound that is used, age, body weight, general health, gender, diet, administration time, route of administration, excretion rate, drug formulation, and severity of the particular disease to be prevented or treated. The pharmaceutical composition according to the present invention may be formulated into pills, sugar-coated tablets, capsules, liquids, gels, syrups, slurries, and suspensions.

In a preferred embodiment, for parenteral administration, the pharmaceutical composition of the present invention is prepared as an aqueous solution. Preferably, a physically suitable buffer solution such as Hank's solution, Ringer's solution, or physically buffered saline is used. An aqueous injection suspension may be added with a substrate capable of increasing the viscosity of the suspension, such as sodium carboxymethyl cellulose, sorbitol, or dextran. In addition, the suspension of the active ingredient may be prepared in the form of a suitable oily injection suspension. Suitable lipophilic solvents or carriers include fatty acids such as sesame oil or synthetic fatty acid esters such as ethyl oleate, triglycerides, or liposomes. Non-lipid polycationic amino polymers may also be used as carriers. Optionally, the suspension may employ a suitable stabilizer or agent to increase the solubility of the compound and to prepare a high-concentration solution.

As used herein, the term “treatment” refers to any action in which symptoms of the above diseases are ameliorated or eliminated by administration of the pharmaceutical composition including the recombinant vector containing the cDNA of soluble VEGF receptor variant (sVEGFRv-1 cDNA).

In the present invention, the term “vector” refers to a DNA product containing a DNA sequence operably linked to a suitable control sequence capable of expressing DNA in a suitable host. The vector may be a plasmid, a phage particle, or simply a potential genomic insert. When transformed into an appropriate host, the vector may replicate and function independently of the host genome, or may often be integrated with the genome itself. Since a plasmid is currently the most commonly used form of vector, “plasmid” and “vector” may be sometimes used interchangeably in the context of the present invention. For the purposes of the present invention, it is preferred to use a plasmid vector. A typical plasmid vector that may be used for this purpose has a structure including (a) a replication origin that allows efficient replication so as to include hundreds of plasmid vectors per host cell, (b) an antibiotic resistance gene that enables selection of host cells transformed with a plasmid vector, and (c) a restriction enzyme cleavage site into which a foreign DNA fragment may be inserted. Even when an appropriate restriction enzyme cleavage site is not present, the vector and foreign DNA may be easily ligated using a synthetic oligonucleotide adapter or linker according to a typical method.

After ligation, the vector has to be transformed into an appropriate host cell. In the present invention, the preferred host cell is a prokaryotic cell. Suitable prokaryotic host cells include E. coli DH5α, E. coli JM101, E. coli K12, E. coli W3110, E. coli X1776, E. coli XL-1Blue (Stratagene), E. coli B, E. coli B21, and the like. However, E. coli strains such as FMB101, NM522, NM538, and NM539, other prokaryotic species and genera, etc. may also be used. In addition to the aforementioned E. coli, strains of the genus Agrobacterium such as Agrobacterium A4, Bacillus strains such as Bacillus subtilis, other Enterobacteriaceae such as Salmonella typhimurium or Serratia marcescens, and various strains of the genus Pseudomonas may be used as host cells.

Transformation of prokaryotic cells may be readily accomplished using a calcium chloride method described in section 1.82 of Sambrook et al., supra. Alternatively, electroporation (Neumann et al., EMBO J., One:841, 1982) may also be used for transformation of these cells.

In the present invention, the term “expression control sequence” refers to a DNA sequence essential for the expression of a coding sequence operably linked to a particular host organism. This control sequence includes promoters for transcription, any operator sequences for regulating such transcription, sequences encoding suitable mRNA ribosome-binding sites, and sequences for regulating the termination of transcription and translation. For example, control sequences suitable for prokaryotes include promoters, any operator sequences, and ribosome-binding sites. Eukaryotic cells include promoters, polyadenylation signals, and enhancers. The factor most strongly affecting the expression level of a gene in the plasmid is a promoter. As the promoter for high expression, an SRα promoter, a promoter derived from cytomegalovirus, and the like are preferably used. In order to express the DNA sequence of the present invention, any of a wide variety of expression control sequences may be used in the vector. Useful expression control sequences include, for example, early and late promoters of SV40 or adenovirus, lac system, trp system, TAC or TRC system, T3 and T7 promoters, major operator and promoter regions of phage lambda, control regions of fd code proteins, promoters of 3-phosphoglycerate kinase or other glycolytic enzymes, promoters of phosphatase, such as Pho5, promoters of yeast alpha mating systems, other sequences known to control gene expression in prokaryotic or eukaryotic cells or viruses, and various combinations thereof. The T7 promoter may be useful to express the protein of the present invention in E. coli.

A nucleic acid is said to be “operably linked” when it is placed into a functional relationship with another nucleic acid sequence. It may be a gene(s) and a control sequence(s) linked in such a way that appropriate molecules (e.g. transcriptional activation proteins) enable gene expression when binding to the control sequence(s). For example, DNA for a pre-sequence or secretory leader is operably linked to DNA for a polypeptide when expressed as a preprotein that participates in secretion of the polypeptide, a promoter or enhancer is operably linked to a coding sequence when affecting the transcription of the sequence, a ribosome-binding site is operably linked to a coding sequence when affecting the transcription of the sequence, or the ribosome-binding site is operably linked to a coding sequence when positioned to facilitate translation. In general, the term “operably linked” means that the linked DNA sequences are contiguous, and, in the case of a secretory leader, contiguous and in the same reading frame. However, enhancers do not have to be contiguous. Linkage of these sequences is accomplished by ligation at convenient restriction enzyme sites. If such sites do not exist, synthetic oligonucleotide adapters or linkers are used in accordance with conventional practice.

As used herein, the term “expression vector” typically refers to a recombinant carrier into which a heterologous DNA fragment is inserted, and generally means a fragment of double-stranded DNA. Here, the heterologous DNA is xenogenous DNA that is not naturally found in the host cell. Once in the host cell, an expression vector may replicate independently of the host chromosomal DNA, and several copies of the vector and inserted (heterologous) DNA thereof may be produced.

As is well known in the art, in order to increase the expression level of a transfected gene in a host cell, the corresponding gene must be operably linked to a transcriptional and translational expression control sequence that functions in the selected expression host. Preferably, the expression control sequence and the corresponding gene are included in a single expression vector including both the bacterial selection marker and the replication origin.

The host cell transformed or transfected with the expression vector described above constitutes a further aspect of the present invention. As used herein, the term “transformation” refers to introduction of DNA into a host such that DNA becomes replicable either as an extrachromosomal factor or through chromosomal integration. As used herein, the term “transfection” means that an expression vector is accommodated by the host cell, regardless of whether or not any coding sequence is actually expressed.

A better understanding of the present invention may be obtained through the following examples. These examples are merely set forth to illustrate the present invention, and are not to be construed as limiting the scope of the present invention, as will be apparent to those skilled in the art.

### Example 1: Construction of rAAV2-sVEGFRv-1

A soluble VEGF receptor-1 variant (sVEGFRv-1) was designed using a human vascular endothelial growth factor receptor (VEGFR) 1 gene (XM_017020485.1, NCBI reference sequence, NIH).

As shown in FIG. 1, sVEGFRv-1 of the present invention is a variant having 6 immunoglobulin-like domains having binding sites with VEGF and PIGF and 31 N-termini identical to FLT-1, and FLT-1s, which is a naturally occurring variant of FLT-1, has 6 immunoglobulin-like domains and a tail composed of 37 amino acids different from FLT-1, and thus the variants have configurations different from each other (Kendall, R. L. and Thomas, K. A. Proc. Natl. Acad. Sci. USA 90, 10705-10709, 1993).

In order to construct pAAV2-sVEGFRv-1, the sVEGFRv-1 spanning nucleotide position 282-2253 (total 1972 bp: SEQ ID NO: 1) was inserted into a pAAV-F.IX cis plasmid containing a CMV promoter, an SV40 polyadenylation signal, and two ITRs (U.S. Pat. No. 6,093,392). Also, rAAV2-GFP was constructed by inserting a GFP gene into the same plasmid.

The sVEGFRv-1 insertion fragment was constructed through PCR using the following primer pair.

sVEGFRv-1 F1:
(SEQ ID NO: 3)
AAGGTACCGC CACCATGGTCAGCTACTGGGACA
sVEGFRv-1 R3:
(SEQ ID NO: 4)
CGCTCGAGCTA TCTGATTGTAATTTCTTTCTTCTG

In order to construct a recombinant rAAV2-sVEGFRv-1 vector and rAAV2-eGFP for use in gene therapy, in addition to the pAAV-sVEGFRv-1 constructed above, an AAV rep-cap plasmid DNA expressing the AAV rep part and the cap part (pAAV-R2C2 plasmid, Stratagene Co., USA) and an adenovirus helper plasmid (pHelper plasmid, Stratagene Co., USA) are required. All of the above three types of plasmid DNA (pAAV-sVEGFRv-1, pAAV-R2C2, and pHelper) were transfected into HEK293 (human embryonic kidney 293; ATCC CRL-1573) cells and then cultured for 72 hours, after which the HEK293 cells were harvested and sonicated, recombinant AAV (rAAV) particles were subjected to CsCl density gradient centrifugation three times, and portions in which RI (refractive index) was primarily measured to be 1.37-1.42 g/mL and in which RI was secondarily measured to be 1.35-1.43 g/mL were collected and isolated to obtain a rAAV2-sVEGFRv-1 vector. Also, rAAV2-eGFP was obtained in the same manner as above.

### Example 2: Confirmation of Expression of rAAV2-sVEGFRv-1 in Retina

The in-vivo expression of rAAV2-sVEGFRv-1 constructed in Example 1 was confirmed as follows.

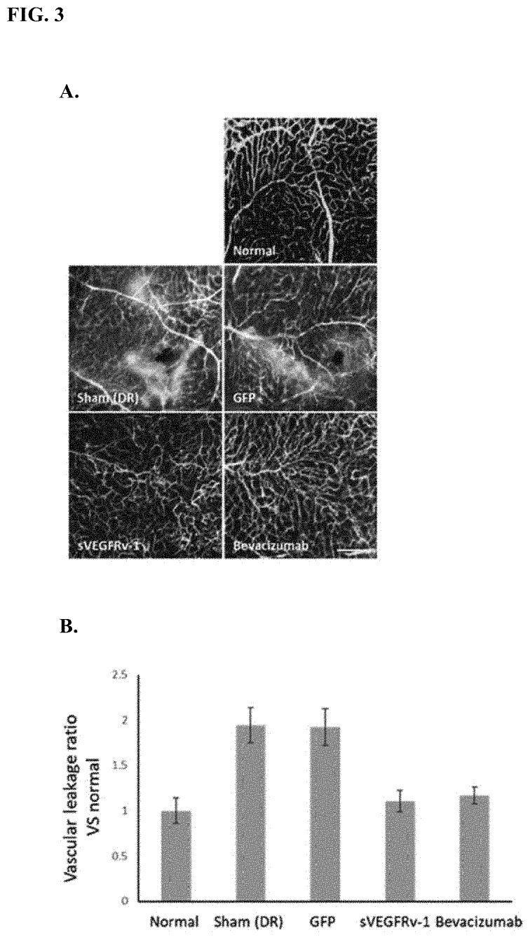

After administering rAAV2-sVEGFRv-1 into both eyes of normal mice (C57BL, 6-7 weeks old) through intravitreal (IV) injection at 5×10⁷ vg/eye, the eyes were extracted therefrom a week later. Thereafter, retinal tissue specimens were manufactured and then subjected to immunostaining for the human soluble VEGF receptor, and expression of the therapeutic gene was confirmed. Here, the antibody used for immunostaining was an anti-human VEGF receptor (anti-Human VEGF-R1/FLT, purified-monoclonal antibody, Invitrogen). The vitreous body was isolated and confirmed through ELISA (Quantikine ELISA Human VEGF R1/Flt-1 Immunoassay, SVR100C, R&D SYSTEMS) for the human soluble VEGF receptor. The average of the measurement results was 4.5±6.7 pg in the normal group, and was observed to be 59.6±41.4 pg in the eye injected with rAAV2-sVEGFRv-1 (5×10⁷ vg/eye) (n=6, p<0.01). The results thereof are shown in FIG. 2.

### Example 3: Confirmation of Vascular Leakage Inhibitory Effect in Diabetic Retinopathy Model Using Dextran-FITC

A diabetic retinopathy animal model for observing the in-vivo therapeutic effect of rAAV2-sVEGFRv-1 constructed in Example 1 was prepared as follows.

Streptozotocin was prepared at a concentration of 150 mg/kg in a 0.1 M citrate buffer and injected once to mice (C57BL, 6-7 weeks old) through intraperitoneal administration, and after 1 week, blood glucose was measured following 8 hours of fasting, during which unlimited drinking water was provided, so an animal model with hyperglycemia (high blood glucose measured to be 300 or more using a blood glucose meter) was used. Four weeks after streptozotocin injection, rAAV2-sVEGFRv-1 was administered to both eyes at a dose of 5×10⁷ vg/eye through intravitreal (IV) injection, after which the therapeutic efficacy thereof on diabetic retinopathy was analyzed in diabetes-induced disease animals.

For measurement of therapeutic efficacy, retinal vascular leakage was analyzed through fluorescence fundus imaging. Briefly, in order to evaluate vascular leakage in the retina, dextran-FITC was injected intraperitoneally, the eyes were extracted, and each retina was isolated, spread on a glass slide, covered with a cover glass, and observed using a fluorescence microscope to comparatively analyze the therapeutic effect between the control group and the group administered with the therapeutic vector.

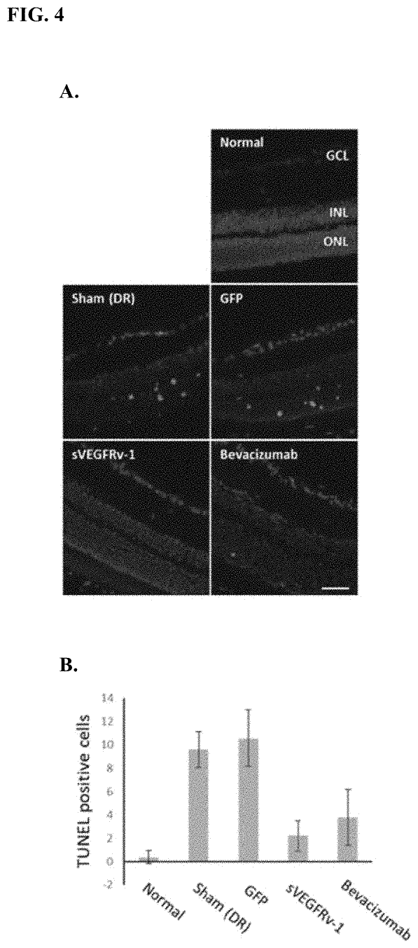

In the diabetic retinopathy model, two control material groups (a group administered with a vehicle (Sham (DR)) and a group administered with a rAAV2-eGFP vector at 5×10⁷ vg/eye (GFP)), a positive control group (Avastin, 1 μL of bevacizumab administered to both eyes at a concentration of 25 mg/ml), and a group administered with a rAAV2-sVEGFRv-1 therapeutic vector received intravitreal (IV) administration, after which vascular leakage was observed. As shown in FIG. 3, compared to the normal group, the vascular leakage ratio was observed to be 1.94±0.19 in the control group (Sham), 1.93±0.2 in the group administered with the GFP vector (GFP), 1.11±0.12 in the group administered with the rAAV2-sVEGFRv-1 therapeutic vector, and 1.17±0.09 in the group administered with Avastin (bevacizumab), and when compared with the control groups (Sham, GFP), it was confirmed that vascular leakage was inhibited by administration of the rAAV2-sVEGFRv-1 therapeutic vector (p<0.01).

### Example 4: Observation of Retinal Neurodegeneration in Hyperglycemia-Induced Diabetic Retinopathy Model

In order to confirm retinal neurodegeneration in a mouse model of diabetic retinopathy induced by hyperglycemia through the method of Example 2, the therapeutic vector rAAV2-sVEGFRv-1 was injected thereto, and after a predetermined period of time, such as 1 month or so, the eyes were extracted and then frozen sections thereof were taken and observed. For observation of cell death, dying cells were stained using TUNEL staining.

In the diabetic retinopathy model, two control material groups (a group administered with a vehicle and a group administered with a rAAV2-eGFP vector at 5×10⁷ vg/eye), a positive control group (Avastin, 1 μL of bevacizumab administered to both eyes at a concentration of 25 mg/mL), and a group administered with a rAAV2-sVEGFRv-1 therapeutic vector received intravitreal (IV) administration, after which cell death was analyzed.

Consequently, as shown in FIG. 4, it was confirmed that there was a therapeutic effect of inhibition of retinal neurodegeneration due to administration of the rAAV2-sVEGFRv-1 therapeutic vector.

### Example 5: Preservation of Retinal Ganglion Cells and Observation of Glial Cell Activity in Retinal Degeneration Caused by Hyperglycemia

In order to confirm retinal degeneration in a mouse model of diabetic retinopathy induced by hyperglycemia through the method of Example 2, the survival of retinal ganglion cells and the activity of glial cells were observed. The retinal ganglion cells were confirmed using a NeuN antibody, and glial cells were observed through immunostaining using a GFAP antibody. For staining with NeuN and GFAP, cryosectioned retinal tissue was used, immunostained, and then observed using a fluorescence microscope.

In the diabetic retinopathy model, two control material groups (a group administered with a vehicle and a group administered with a rAAV2-eGFP vector at 5×10⁷ vg/eye), a positive control group (Avastin, 1 μL of bevacizumab administered to both eyes at a concentration of 25 mg/ml), and a group administered with a rAAV2-sVEGFRv-1 therapeutic vector received intravitreal (IV) administration, after which the results thereof were comparatively analyzed.

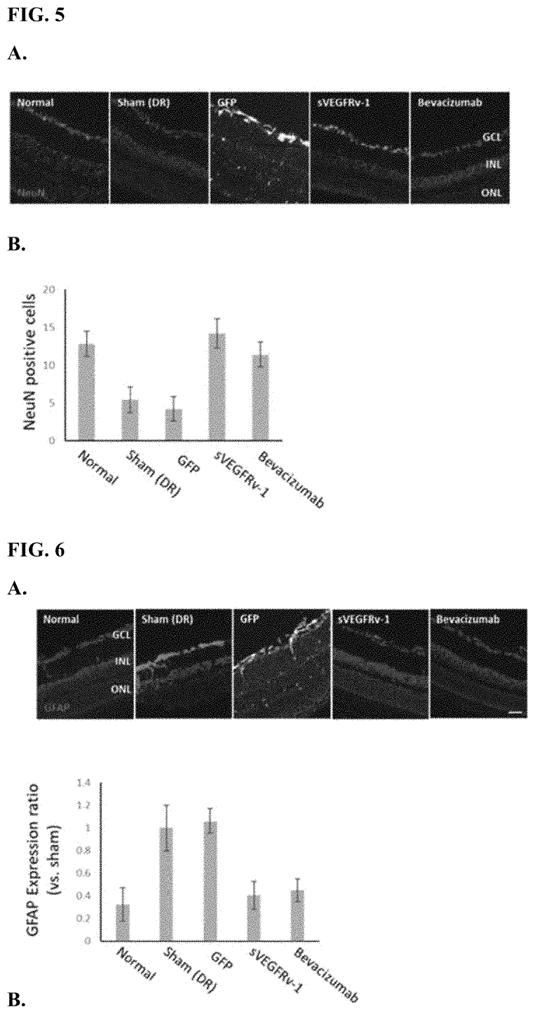

Consequently, as shown in FIGS. 5 and 6, it was confirmed that the cells stained with NeuN were decreased in the control groups compared to the normal retina, and the preservation of NeuN positive cells due to administration of rAAV2-sVEGFRv-1 was confirmed. In addition, the extent of staining with GFAP was low compared to the control groups, which means that the activity of glial cells was inhibited by administration of rAAV2-sVEGFRv-1.

### Example 6: Isolation of Retinal Blood Vessels to Observe Retinal Vascular Degeneration

In order to confirm retinal vascular degeneration in a mouse model of diabetic retinopathy induced by hyperglycemia through the method of Example 2, retinal blood vessels were isolated, and changes in retinal blood vessels and degeneration of pericytes such as endothelial cells and acellular capillaries were observed. The retinal blood vessels were isolated using a trypsin digestion method, and the isolated blood vessels were subjected to H&E staining to observe pericytes and acellular capillaries.

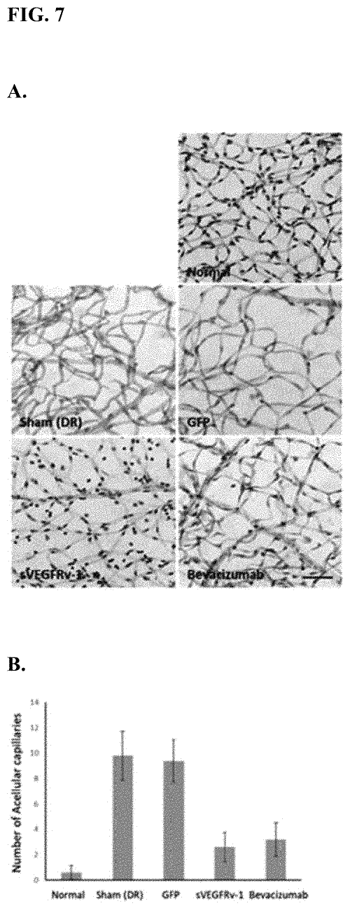

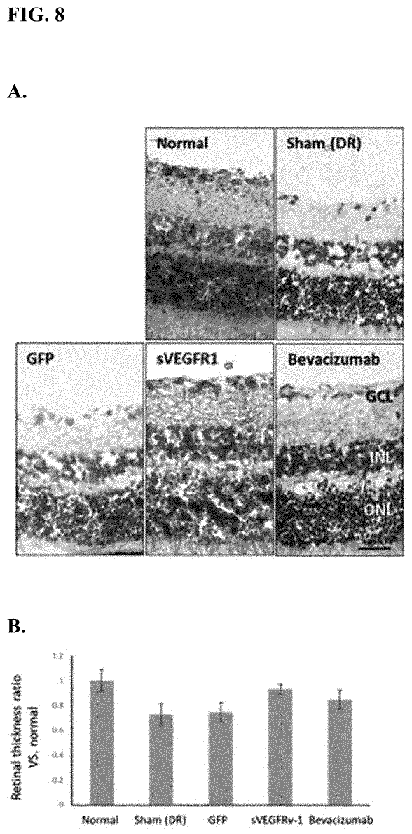

In the diabetic retinopathy model, two control material groups (a group administered with a vehicle and a group administered with a rAAV2-eGFP vector at 5×10⁷ vg/eye), a positive control group (Avastin, 1 μL of bevacizumab administered to both eyes at a concentration of 25 mg/ml), and a group administered with a rAAV-sVEGFRv-1 therapeutic vector received intravitreal (IV) administration, after which degeneration of blood vessels and pericytes was observed. Consequently, as shown in FIG. 7, it was confirmed that vascular degeneration was inhibited by administration of the rAAV-sVEGFRv-1 therapeutic vector.

### Example 7: Retinal Tissue Staining to Observe Retinal Degeneration

In order to confirm retinal vascular degeneration in a mouse model of diabetic retinopathy induced by hyperglycemia through the method of Example 2, changes in retinal tissue were observed 6 months after induction of the disease. Retinal staining was performed by fixing the eye, removing the crystalline lens and cornea, manufacturing a frozen section, and then conducting H&E staining.

The stained retinal tissue slides were observed using a microscope, and changes in retinal tissue were analyzed by observing changes in retinal thickness. The thickness of the retina was analyzed by measuring changes in the thickness of the inner nuclear layer and the inner flexiform layer.

In the diabetic retinopathy model, two control materials (a group administered with a vehicle and a group administered with a rAAV2-eGFP vector at 5×10⁷ vg/eye), a positive control group (Avastin, 1 μL of bevacizumab administered to both eyes at a concentration of 25 mg/ml), and a group administered with a rAAV2-sVEGFRv-1 therapeutic vector were compared for thickness of the retinal tissue. Consequently, it was confirmed that there was a retinal protective effect in the group administered with the rAAV2-sVEGFRv-1 therapeutic vector, and as shown in FIG. 8, it was confirmed that degeneration of retinal nerve tissue was inhibited by administration of the rAAV2-sVEGFRv-1 therapeutic vector.

### INDUSTRIAL APPLICABILITY

According to the present invention, diabetic retinopathy can be treated through a single administration, and compared to conventional treatment methods that require monthly intravitreal injection, patient pain, ocular damage, and risk of infection can be alleviated, and treatment costs can be reduced.

Although specific embodiments of the present invention have been disclosed in detail above, it will be obvious to those skilled in the art that the description is merely of preferable exemplary embodiments and is not to be construed as limiting the scope of the present invention. Therefore, the substantial scope of the present invention will be defined by the appended claims and equivalents thereof.

[Sequence List Free Text]

An electronic file is attached.

1. A method of treating or preventing diabetic retinopathy comprising administering a recombinant vector containing a cDNA of a soluble VEGF receptor variant.

2. The method according to claim 1, wherein the soluble VEGF receptor variant has an amino acid sequence represented by SEQ ID NO: 2.

3. The method according to claim 1, wherein the recombinant vector is an adeno-associated virus (AAV) vector.

4. The method according to claim 1, wherein the recombinant vector is rAAV2 or another rAAV serotype.

5. The method according to claim 1, wherein the recombinant vector is administered once every 2-3 years.
